(19) United States
(12) Patent Application Publication
(10) Pub. No.: US 2014/0100400 A1
(43) Pub. Date: Apr. 10, 2014

(54) METHOD FOR PREPARING PHENYLCYCLOHEXANE

(71) Applicant: BASF SE, Ludwigshafen (DE)

(72) Inventor: Martin BOCK, Ludwigshafen (DE)

(73) Assignee: BASF SE, Ludwigshafen (DE)

(21) Appl. No.: 14/041,380

(22) Filed: Sep. 30, 2013

Related U.S. Application Data
(60) Provisional application No. 61/709,355, filed on Oct. 4, 2012.

(51) Int. Cl. C07C 5/11

(52) U.S. Cl. CPC C07C 5/11
USPC 585/265; 585/268

(57) ABSTRACT

A method for preparing a phenylcyclohexane of formula I

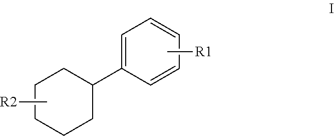

by hydrogenation of a biphenyl of formula II

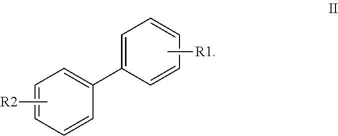

with hydrogen in the presence of Raney nickel,
where R1 and R2 both have the same meaning in formulas I and II and independently of one another are hydrogen atoms, C1- to C10-alkyl groups or phenyl groups,
wherein the hydrogenation takes place in the presence of 0 to 20 parts by weight of water to 100 parts by weight of Raney nickel.

The invention relates to a method for preparing a phenylcyclohexane of formula I

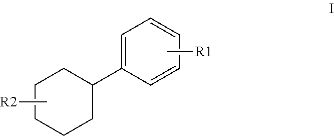

by hydrogenation of a biphenyl of formula II

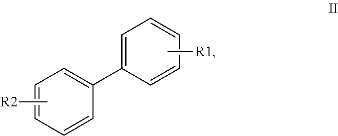

with hydrogen in the presence of Raney nickel, where R1 and R2 both have the same meaning in formulas I and II and independently of one another are hydrogen atoms, C1- to C10-alkyl groups or phenyl groups,

wherein the hydrogenation takes place in the presence of 0 to 20 parts by weight of water to 100 parts by weight of Raney nickel.

Phenylcyclohexane has industrial significance as a starting material or intermediate in chemical syntheses; phenylcyclohexane is also particularly used as a solvent, heat carrier or heat transfer medium.

The preparation of phenylcyclohexane may take place by hydrogenation of biphenyl. For this purpose, various methods are to be found in the prior art.

WO2012/059387 A1 describes a continuous method for preparing phenylcyclohexane by hydrogenation of biphenyl over a catalyst which comprises nickel, zirconium, copper and molybdenum. The conversion achieved is, at maximum, 95%.

The use of Raney nickel as a catalyst for hydrogenating biphenyl is known from EP-A 13799602 and Tetrahedron Letters 2000, Vol. 41, 5865-5868.

By reason of its reactivity, Raney nickel is generally in the form of a suspension in a suitable solvent and is used in this form. Tetrahedron Letters 2000, Vol. 41, 5865-5868 describes the use of aqueous Raney nickel for the hydrogenation of phenylcyclohexane. Water does not mix with the phenylcyclohexane obtained and may be separated off as a separate phase. The yield of phenylcyclohexane is 91 to 95%.

Based on the prior art, the object of the present invention consists in providing as simple and economical a method as possible for preparing phenylcyclohexane. The space-time yields of phenylcyclohexane should be as high as possible. Phenylcyclohexane should be obtained with high selectivity and purity.

Accordingly, the method defined above has been found.

In the method a phenylcyclohexane of formula I is prepared

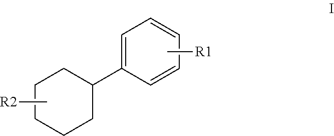

by hydrogenation of a biphenyl of formula II.

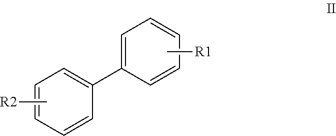

The carbon atoms of the phenyl rings in formula II are each substituted by a hydrogen atom; only one carbon atom of each of the phenyl rings carries the R1 or R2 residue as a substituent. R1 and R2 in formula II may be linked to any desired carbon atom of the respective phenyl ring, e.g. R1 and R2 independently of one another may be located in the ortho-, meta- or para- position (based on the carbon atom linked to the second ring).

Preferably, R1 and R2, independently of one another, are hydrogen atoms or C1- to C4-alkyl groups; in particular, R1 and R2 independently of one another represent a hydrogen atom or a methyl group.

R1 and R2 are very particularly preferably hydrogen atoms; correspondingly, the compound of formula II is biphenyl.

The meanings of R1 and R2 in the biphenyl of formula II (starting material) and the phenylcyclohexane of formula I (product) are identical. Correspondingly, the product is very particularly preferably phenylcyclohexane (R1 and R2 are hydrogen atoms).

The hydrogenation takes place with elemental hydrogen in the presence of Raney nickel. Raney nickel is a solid—generally in the form of grains or powder—which consists of more than 50% by weight, particularly more than 80% by weight, of nickel. Due to the process of preparation Raney nickel has a porous structure and consequently a large surface area. The starting material for the preparation of Raney nickel is typically a nickel-aluminum alloy, out of which aluminum is leached, e.g. by sodium or potassium hydroxide solution. Raney nickel may therefore still comprise aluminum depending on the preparation and completeness of the removal of the aluminum. The preparation of Raney nickel is described, for example, in U.S. Pat. No. 1,628,190.

By reason of its high nickel content and high surface area, Raney nickel is very reactive and starts to burn on contact with oxygen. For this reason, Raney nickel is mixed with a solvent and handled and used in the form of a solvent-containing suspension.

Suitable solvents here are water and also various organic solvents. In the method according to the invention water is used as the solvent for the Raney nickel. Thus, the Raney nickel is in the form of an aqueous suspension. The water obtained in the reaction does not mix with the reaction product of formula I. Therefore, the water may be readily separated off after the reaction by phase separation.

Raney nickel is preferably used in amounts of 0.1 to 5 parts by weight (solid, i.e. without solvent) to 100 parts by weight of the biphenyl of formula II (starting material). Raney nickel is particularly preferably used in amounts of 0.1 to 2.5, very particularly preferably in amounts of 0.5 to 1.5 parts by weight, to 100 parts by weight of the biphenyl of formula II.

A commercially available Raney nickel catalyst is e.g. Actimet M® from BASF.

In the method according to the invention, the water is largely or completely removed before the hydrogenation and the hydrogenation takes place in the presence of only 0 to 20 parts by weight of water, preferably from 0 to 5 parts by weight and very particularly preferably from 0 to 1 part by weight of water, to 100 parts by weight of Raney nickel (solid). In a particular embodiment, the water content is less than 100 ppm water, based on Raney nickel.

To carry out the method according to the invention, biphenyl is brought into contact with the catalyst in a reaction vessel and hydrogenated with gaseous hydrogen. Further feedstocks such as solvents are not required. The hydrogenation is thus preferably conducted in the absence of further feedstocks, in particular in the absence of other solvent.

For the hydrogenation, gaseous hydrogen is fed into the reaction vessel, which comprises the biphenyl of formula II and the catalyst. Hydrogen may also be used mixed with other gases, e.g. inert gases such as nitrogen or helium; preferably, however, only hydrogen is used.

Preferably, the hydrogenation takes place in the absence of oxygen.

The hydrogenation is preferably carried out at a pressure of 10-100 bar, particularly preferably at a pressure of 30-60 bar. The reaction vessel is preferably purged of oxygen by flushing with hydrogen or an inert gas and then the pressure adjusted with the gas used, preferably exclusively hydrogen. During the hydrogenation, hydrogen is preferably supplied to replace the hydrogen consumed, i.e. the hydrogen pressure is preferably kept constant.

The hydrogenation takes place preferably at 70 to 200° C. and particularly preferably at 100 to 160° C. The temperature refers to the reactor contents, i.e. the temperature in the reaction mixture.

The hydrogenation may be conducted in continuous, semi-continuous or discontinuous mode. In the continuous mode, all feedstocks (hydrogen and biphenyl of formula II) are continuously fed into the reaction vessel and the products continuously removed. In the semi-continuous mode only single feedstocks are continuously fed in.

The hydrogenation is preferably carried out discontinuously, i.e. batchwise; for this purpose, the starting materials (hydrogen and biphenyl of formula II) are placed in the reaction vessel, and the hydrogenation is carried out and discontinued after the desired reaction time or after reaching the desired conversion.

The duration of the hydrogenation may be determined by the hydrogen consumption, which may be monitored e.g. by a mass flowmeter. In addition, the course of the reaction may be monitored by sampling (e.g. GC or refractive index). On reaching the desired conversion, the hydrogen feed is stopped and the reaction vessel cooled.

The hydrogenation is preferably discontinued as soon as more than 95% by weight, particularly more than 98.5% by weight, of the biphenyl used has been converted.

The catalyst may be separated from the product obtained by filtration or sedimentation. The catalyst is preferably separated by filtration.

The product has a phenylcyclohexane content of more than 97% by weight, preferably more than 98.5% by weight, based on the biphenyl of formula II used.

The content of unreacted starting material (biphenyl of formula II) is preferably less than 1% by weight, particularly less than 0.5% by weight.

A by-product formed may be a compound in which both phenyl rings of the biphenyl of formula II are completely hydrogenated (bicyclohexyl); the content of bicyclohexyl is also preferably less than 1%, in particular less than 0.5%.

The Raney nickel separated from the product after the hydrogenation may be used again; preferably it is reused for the hydrogenation, according to the invention, of a biphenyl of formula II to a phenylcyclohexane of formula I.

In a preferred embodiment of the repeated hydrogenation of a biphenyl of formula II to a phenylcyclohexane of formula I, Raney nickel is used for the first time as an aqueous suspension and the aforementioned method, according to the invention, is first carried out by in repeated usage of the recovered Raney nickel.

Thus, the present application also provides a method for repeated preparation of a phenylcyclohexane of formula I by hydrogenation of a biphenyl of formula II with hydrogen in the presence of Raney nickel, wherein
the first hydrogenation takes place in the presence of more than 20 parts by weight of water to 100 parts by weight of Raney nickel,
water is removed from the product mixture obtained and Raney nickel is separated off and
at least one further hydrogenation takes place in the presence of 0 to 20 parts by weight of water to 100 parts by weight of the separated and reused Raney nickel.

The hydrogenation in the presence of water takes place as described above, with the exception that Raney nickel is used as an aqueous suspension. Conventional aqueous suspensions of Raney nickel have a water content of e.g. 50 to 150 parts by weight of water to 100 parts by weight of Raney nickel. These aqueous suspensions may be used for the first hydrogenation without separation from water.

After the first hydrogenation the water present as a separate phase may be removed by phase separation. The now anydrous Raney nickel may be used henceforth for the further hydrogenations according to the invention, i.e. in the presence of 0 to 20 parts by weight of water, preferably 0 to 5 parts by weight, particularly 0 to 1 part by weight of water, particularly preferably less than 100 ppm of water, to 100 parts by weight of Raney nickel. The separation of the Raney nickel takes place preferably after the first and the subsequent hydrogenations by filtration (see above). The filtration is preferably carried out with exclusion of oxygen. The Raney nickel filtered off may then be fed back into the reaction vessel by rinsing the filter with product (phenylcyclohexane of formula I) or starting material (biphenyl of formula II).

In the case of the method of repeated preparation of phenylcyclohexane of formula I, further hydrogenations may also take place in the presence of water. For example, after the first hydrogenation, the second hydrogenation may also still take place in the presence of water. For this, the Raney nickel recovered from the previous hydrogenation may be rinsed back into the reaction vessel with water.

The third hydrogenation and all further hydrogenations take place preferably in the absence of water. Particular preference is given to the second hydrogenation and all further hydrogenations taking place in the absence of water.

The hydrogenation may be repeated, after the first hydrogenation, at least twice, in particular at least five times, particularly preferred at least 10 times, with the above-stated conversions to phenylcyclohexane. Repeated hydrogenations with the same Raney nickel are possible e.g. 5 to 100 or 5 to 50 or 10 to 50 times, only if the first hydrogenation or the first two hydrogenations have been carried out in the presence of water.

The method according to the invention is a simple and economical method for preparing phenylcyclohexanes of formula I. The space-time yields of phenylcyclohexane of formula I are very high; the yields of phenylcyclohexane are preferably greater than 97% by weight, in particular greater than 98% by weight, based on the biphenyl of formula II used. The method according to the invention is particularly suitable as part of a recycling process. The method according to the invention allows a frequent repetition of the hydrogenation by using the same Raney nickel of consistent quality.

### EXAMPLE

Biphenyl is the compound of formula II where R1 and R2 are hydrogen.

Example of the preparation of Raney nickel

In an induction oven, 2 kg of a mixture consisting of 49.3 percent by weight of nickel, 49.3 percent by weight of aluminum and 1.4 percent by weight of an aluminum oxide-graphite mixture were heated with 80 percent by weight of carbon. Due to the exothermic reaction, temperatures of approx. 1500° C. were reached. On completion of the reaction, the temperature is lowered to 1300° C. and heat treatment is effected for 20 minutes. Subsequently the melt is cooled to 25° C. over 2 hours and the melt mass is ground to the desired particle size. The activation of the ground alloy takes place by stirring for 2 hours in 25% aqueous potassium hydroxide solution at temperatures between 70 and 80° C. Subsequently the Raney alloy is first washed with dilute hydrochloric acid and then with water to substantially free it of alkali metal and halogen, and the active Raney nickel catalyst obtained is stored under water (50 percent by weight).

Analysis: GC analysis was carried out according to the following method: 30 m DB-WAX, ID.: 0.25 mm, FT. 0.25 μm, initial temp.: 200° C., det. temp.: 250° C.; start 80° C.−3° C./min-200° C.−15° C./min to 240° C./20 min isothermal; injection amount: 0.2 1-11; carrier gas N₂; t_{R}=min; t_{R} (biphenyl): 25.2; t_{R} (phenylcyclohexane): 14.6; t_{R} (phenylcyclohexene): 9.1; t_{R} (bicyclohexyl): 7.7.

### Example

First and second hydrogenation in the presence of water, third hydrogenation in the absence of water

First Hydrogenation

In a 30 m³ autoclave were placed 179 kg of a 50 percent by weight aqueous suspension of Raney nickel in water and 3000 kg of molten biphenyl. The hydrogenation was carried out at hydrogen pressure 30 bar and 140° C.-150° C. The progress of the reaction was monitored by gas chromatography (GC). The hydrogenation was discontinued after 36 hours (h). The composition of the product mixture obtained after 36 h was: 0.81% by weight of biphenyl, 98.62% by weight of phenylcyclohexane, 0.07% by weight of phenylcyclohexene and 0.12% by weight of bicyclohexane. After cooling and pressure release the catalyst was separated off on a filter.

Second Hydrogenation

The catalyst separated in the first hydrogenation was rinsed back into the autoclave with 400 kg of water. 7000 kg of biphenyl melt were metered in and heated to 150° C. and 30 bar. In the course of the hydrogenation the pressure was raised to 40 bar and the temperature to 170° C.

The progress of the reaction was monitored by gas chromatography (GC). The hydrogenation was discontinued after 62 hours. The composition of the product mixture obtained after 62 hours was: 0.58% by weight of biphenyl, 98.96% by weight of phenylcyclohexane, 0.03% by weight of phenylcyclohexene and 0.04% by weight of bicyclohexane. After cooling and pressure release the catalyst was separated off on a filter.

Third Hydrogenation

The catalyst separated off in the second hydrogenation was rinsed back into the autoclave with 400 kg of product from the first hydrogenation. 7000 kg of biphenyl melt were metered in and heated to 140° C. and 40 bar. The progress of the reaction was monitored by gas chromatography (GC). The reaction was discontinued after 30 hours. The composition of the product mixture obtained after 30 hours was: 0.08% by weight of biphenyl, 98.93% by weight of phenylcyclohexane, 0.13% by weight of phenylcyclohexene and 0.48% by weight of bicyclohexane. After cooling and pressure release the catalyst was separated off on a filter. After only 30 hours a conversion to phenylcyclohexane which still exceeded that of the second hydrogenation was achieved.

Comparative Example

First, second and third hydrogenation in the presence of water

First Hydrogenation

In a 10 m³ autoclave were placed 120 kg of a 50% aqueous suspension of Raney nickel and 7000 kg of molten biphenyl. The autoclave was heated to 150° C.-175° C. and 30 bar and hydrogenation was effected for 20 h; the progress of the reaction was monitored by GC. The composition of the product mixture obtained after 30 hours was: 0.03% by weight of biphenyl, 98.99% by weight of phenylcyclohexane, 0.17% by weight of phenylcyclohexene and 0.39% by weight of bicyclohexane. After cooling and pressure release the catalyst was separated off on a filter.

Second Hydrogenation

The catalyst from the first hydrogenation was rinsed back into the autoclave with 1000 kg of water. 7000 kg of biphenyl melt were metered in and heated to 150° C. and 40 bar. In the course of the hydrogenation the pressure and temperature were raised to 160° C. and 40 bar. After 24 hours, the reaction was discontinued after GC-monitoring. The composition of the product mixture obtained after 24 hours was 1.41% by weight of biphenyl, 97.84% by weight of phenylcyclohexane, 0.09% by weight of phenylcyclohexene and 0.29% by weight of bicyclohexane. After cooling and pressure release the catalyst was separated off on a filter.

Third Hydrogenation

The catalyst from the second hydrogenation was rinsed back into the autoclave with 1000 kg of water. 7000 kg of biphenyl melt were metered in and heated to 160° C. and 40 bar. Since no hydrogen had yet been consumed after 4 hours, the autoclave was depressurized and cooled to 100° C. A further 120 kg of Raney nickel catalyst were added and once again the autoclave was heated to 140° C. and 40 bar. In the course of the hydrogenation the pressure was raised to 160° C. After 34 h, the reaction was discontinued after GC-monitoring. The composition of the product mixture obtained after 34 hours was: 0.33% by weight of biphenyl, 98.96% by weight of phenylcyclohexane, 0.09% by weight of phenylcyclohexene and 0.24% by weight of bicyclohexane. After cooling and pressure release the catalyst was separated off on a filter.

1. A method for preparing a phenylcyclohexane of formula I: 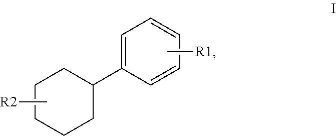
comprising hydrogenating a biphenyl of formula II: 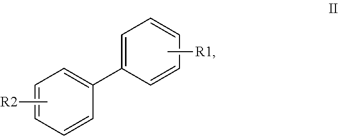
with hydrogen in the presence of Raney nickel,
wherein R1 and R2 are each independently a hydrogen atom, a C1- to C10-alkyl group or a phenyl group, and
the hydrogenating takes place in the presence of from 0 to 20 parts by weight of water to 100 parts by weight of Raney nickel.

2. The method according to claim 1, wherein R1 and R2 are hydrogen atoms.

3. The method according to claim 1, wherein the hydrogenating takes place in the presence of from 0 to 5 parts by weight of water to 100 parts by weight of Raney nickel.

4. The method according to claim 1, wherein the hydrogenating takes place with elemental hydrogen at a pressure of from 10 to 100 bar.

5. The method according to claim 1, wherein the hydrogenating is conducted at a temperature of from 70 to 200° C.

6. The method according to claim 1, wherein the method is a batchwise method.

7. The method according to claim 1, wherein an endpoint of the hydrogenating is determined by consumption of hydrogen.

8. The method according to claim 1, wherein the hydrogenating is discontinued as soon as more than 95% by weight of the biphenyl has reacted.

9. The method according to claim 1, further comprising:
separating the Raney nickel from a product after the hydrogenating, and
performing the hydrogenating again with the separated Raney nickel.

10. A method for repeatedly preparing a phenylcyclohexane of formula I: 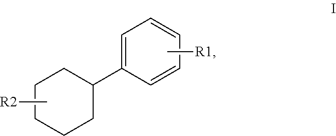
comprising hydrogenating a biphenyl of formula II: 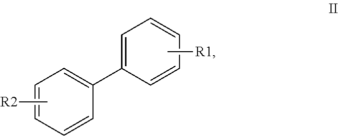
with hydrogen in the presence of Raney nickel,
wherein the method comprises
first hydrogenating in the presence of more than 20 parts by weight of water to 100 parts by weight of Raney nickel,
then removing water from a product mixture obtained and separating the Raney nickel off, and
further hydrogenating in the presence of from 0 to 20 parts by weight of water to 100 parts by weight of a separated and reused Raney nickel.
